(19) United States
(12) Patent Application Publication
(10) Pub. No.: US 2019/0282250 A1
(43) Pub. Date: Sep. 19, 2019

(54) TREATMENT OF VASCULAR LESIONS

(71) Applicant: Boston Scientific Scimed, Inc., Maple Grove, MN (US)

(72) Inventors: Binh C. Tran, Minneapolis, MN (US); Douglas Dean Pagoria, Evergreen, CO (US); Roger W. McGowan, Otsego, MN (US); Christopher Smuk, Champlin, MN (US); Daniel Frank Massimini, Brooklyn Park, MN (US)

(21) Appl. No.: 16/351,186

(22) Filed: Mar. 12, 2019

Related U.S. Application Data
(60) Provisional application No. 62/642,822, filed on Mar. 14, 2018.

(51) Int. Cl. A61B 17/22

(52) U.S. Cl. CPC A61B 17/2202; A61B 2017/22069; A61B 2017/22028

(57) ABSTRACT

An ultrasound catheter is adapted for placement within a blood vessel having a vessel wall and is adapted for treating a vascular lesion within or adjacent the vessel wall. The ultrasound catheter includes an elongate shaft extending from a distal region to a proximal region and an ultrasound transducer that is disposed within the distal region of the elongate shaft, the ultrasound transducer adapted to impart near-field, acoustic pressure waves upon the vascular lesion in order to mechanically modify the vascular lesion.

This application claims the benefit of U.S. Provisional Application No. 62/642,822, filed Mar. 14, 2018, the content of which is herein incorporated by reference in its entirety. This application is co-owned by the owner of U.S. Provisional Application No. 62/642,830, filed Mar. 14, 2018, the content of which is herein incorporated by reference in its entirety.

### TECHNICAL FIELD

The present disclosure pertains to medical devices, and methods for manufacturing and using medical devices. More particularly, the disclosure is directed to devices and methods for softening lesions within or near a vascular lumen.

### BACKGROUND

Many patients suffer from occluded arteries and other blood vessels which restrict blood flow. Occlusions can be partial occlusions that reduce blood flow through the occluded portion of a blood vessel or total occlusions (e.g., chronic total occlusions) that substantially block blood flow through the occluded blood vessel. In some cases, an occlusion may be or otherwise include a calcified lesion that may impact a physician's ability to place a stent, or conduct balloon angioplasty, for example. The calcified lesion may be treated to soften and weaken the calcified lesion, which can make subsequent treatments such as stenting and balloon angioplasty more effective. A need remains for alternate devices and methods for treating calcified lesions.

### SUMMARY

This disclosure provides design, material, manufacturing method, and use alternatives for medical devices. For example, the disclosure is directed to an ultrasound catheter that is adapted for placement within a blood vessel having a vessel wall, the ultrasound catheter for treating a calcified lesion within or adjacent the vessel wall. The ultrasound catheter includes an elongate shaft that extends from a distal region to a proximal region and an ultrasound transducer that is disposed within the distal region of the elongate shaft and is adapted to impart near-field acoustic pressure waves within the calcified lesion in order to induce fractures in the calcified lesion.

Alternatively or additionally, the ultrasound transducer may be configured to transmit a substantially uniform acoustic pressure over a length of about 5 millimeters to about 60 millimeters at a radial distance of about 1 millimeters to about 8 millimeters as measured from a longitudinal central axis of the elongate shaft. Alternatively or additionally, the ultrasound transducer may be configured to transmit a substantially uniform acoustic pressure over a length of about 10 millimeters to about 60 millimeters at a radial distance of about 1 millimeters to about 8 millimeters as measured from a longitudinal central axis of the elongate shaft.

Alternatively or additionally, the ultrasound transducer may be configured as a planar ultrasound transducer, and may be adapted to output acoustic pressure waves propagating along a primary radial direction.

Alternatively or additionally, the ultrasound transducer may be configured as a plurality of planar ultrasound transducers, and may be adapted to output acoustic pressure waves propagating in a plurality of radial directions.

Alternatively or additionally, the ultrasound transducer may be configured as a cylindrical ultrasound transducer, and may be adapted to output acoustic pressure waves propagating radially outwardly, omnidirectionally from the cylindrical ultrasound transducer.

Alternatively or additionally, the ultrasound transducer may include a plurality of individual ultrasound transducers.

Alternatively or additionally, the plurality of individual ultrasound transducers may be axially spaced apart, with intervening polymeric segments disposed between adjacent ultrasound transducers to impart a degree of flexibility to the ultrasound transducer.

Alternatively or additionally, the plurality of individual ultrasound transducers may be pivotably securable to one another to impart a degree of flexibility to the ultrasound transducer.

Alternatively or additionally, each of the individual ultrasound transducers may be independently electrically driven.

Alternatively or additionally, all of the individual ultrasound transducers may be electrically driven with a common source.

Alternatively or additionally, the ultrasound catheter may also include a fixation structure that is coupled relative to the elongate shaft and moveable between a collapsed configuration that permits the ultrasound catheter to be advanced through a blood vessel and an expanded configuration that anchors the ultrasound catheter within the blood vessel.

Alternatively or additionally, the fixation structure may be mechanically actuatable between the collapsed configuration and the expanded configuration.

Alternatively or additionally, the fixation structure may be constrained in the collapsed configuration for delivery via an outer sheath disposed over the fixation structure and may be self-expanding into the expanded configuration upon removal of the outer sheath.

Another example of the disclosure is an ultrasound device that is adapted for placement within a blood vessel having a vessel wall and for causing mechanical fractures in a calcified lesion within or adjacent the vessel wall. The ultrasound device includes an elongate shaft extending from a distal region to a proximal region and an ultrasound transducer that is disposed within the distal region of the elongate shaft. The ultrasound transducer is adapted to impart unfocused acoustic pressure waves within the calcified lesion in order to induce fractures in the calcified lesion and has an effective length that is at least twice a distance between the ultrasound transducer and the calcified lesion when the ultrasound device is disposed proximate the calcified lesion.

Alternatively or additionally, the ultrasound transducer may have an effective length that is at least three times a distance between the ultrasound transducer and the calcified lesion when the ultrasound device is disposed proximate the calcified lesion.

Alternatively or additionally, the ultrasound transducer may have an effective length that is longer than a length of the calcified lesion.

Alternatively or additionally, the ultrasound device may further include a fixation element that is coupled to the elongate shaft and is adapted to releasably secure the ultrasound device within a blood vessel.

Another example of the disclosure is an ultrasound catheter that is adapted for placement within a blood vessel having a vessel wall, the ultrasound catheter for treating a vascular lesion within or adjacent the vessel wall. The ultrasound catheter includes an elongate shaft extending from a distal region to a proximal region and a fixation structure that is coupled relative to the elongate shaft and moveable between a collapsed configuration that permits the ultrasound catheter to be advanced through a blood vessel and an expanded configuration that anchors the ultrasound catheter within the blood vessel. An ultrasound transducer is disposed within the distal region of the elongate shaft and is adapted to impart near-field acoustic pressure waves upon the vascular lesion in order to mechanically modify the vascular lesion and increase distensibility of the blood vessel.

Alternatively or additionally, the fixation structure may be mechanically actuatable between the collapsed configuration and the expanded configuration.

Alternatively or additionally, the fixation structure may be self-expanding.

The above summary of some embodiments is not intended to describe each disclosed embodiment or every implementation of the present disclosure. The Figures, and Detailed Description, which follow, more particularly exemplify these embodiments.

### BRIEF DESCRIPTION OF THE DRAWINGS

The invention may be more completely understood in consideration of the following detailed description of various embodiments of the invention in connection with the accompanying drawings, in which:

FIG. 1 is a schematic illustration of a near-field ultrasound field created by an ultrasound catheter in accordance with the disclosure;

FIG. 2 is a schematic illustration of an ultrasound transducer system in accordance with the disclosure;

FIG. 3 is a schematic illustration of an ultrasound transducer system in accordance with the disclosure;

FIG. 4 is a schematic illustration of an ultrasound transducer system in accordance with the disclosure;

FIG. 5 is a schematic cross-sectional view of a portion of an ultrasound catheter in accordance with the disclosure;

FIG. 6 is a schematic cross-sectional view of a portion of an ultrasound catheter in accordance with the disclosure;

FIG. 7 is a schematic cross-sectional view of a portion of an ultrasound catheter in accordance with the disclosure;

FIG. 8 is a schematic cross-sectional view of a portion of an ultrasound catheter in accordance with the disclosure;

FIG. 9 a schematic cross-sectional view of a portion of an ultrasound catheter having a mechanically actuated fixation structure in a collapsed configuration in accordance with the disclosure;

FIG. 10 is a schematic cross-sectional view of the ultrasound catheter of FIG. 9, with the fixation structure in an expanded configuration in accordance with the disclosure;

FIG. 11 is a schematic cross-sectional view of a portion of an ultrasound catheter having a self-expanding fixation structure in a collapsed configuration in accordance with the disclosure;

FIG. 12 is a schematic cross-sectional view of the ultrasound catheter of FIG. 10, with the fixation structure in an expanded configuration in accordance with the disclosure;

FIG. 13 is a schematic cross-sectional view of a portion of an ultrasound catheter having an inflatable fixation structure in an expanded configuration in accordance with the disclosure; and

FIG. 14 is a schematic cross-sectional view of a portion of an ultrasound catheter having an inflatable fixation structure in an expanded configuration in accordance with the disclosure.

While the disclosure is amenable to various modifications and alternative forms, specifics thereof have been shown by way of example in the drawings and will be described in detail. It should be understood, however, that the intention is not to limit the disclosure to the particular embodiments described. On the contrary, the intention is to cover all modifications, equivalents, and alternatives falling within the spirit and scope of the disclosure.

### DETAILED DESCRIPTION

For the following defined terms, these definitions shall be applied, unless a different definition is given in the claims or elsewhere in this specification.

All numeric values are herein assumed to be modified by the term “about,” whether or not explicitly indicated. The term “about” generally refers to a range of numbers that one of skill in the art would consider equivalent to the recited value (i.e., having the same function or result). In many instances, the terms “about” may include numbers that are rounded to the nearest significant figure.

The recitation of numerical ranges by endpoints includes all numbers within that range (e.g. 1 to 5 includes 1, 1.5, 2, 2.75, 3, 3.80, 4, and 5).

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

The following detailed description should be read with reference to the drawings in which similar elements in different drawings are numbered the same. The drawings, which are not necessarily to scale, depict illustrative embodiments and are not intended to limit the scope of the invention.

Many patients suffer from occluded arteries, other blood vessels, and/or occluded ducts or other body lumens which may restrict bodily fluid (e.g. blood, bile, etc.) flow. Occlusions can be partial occlusions that reduce blood flow through the occluded portion of a blood vessel or total occlusions (e.g., chronic total occlusions) that substantially block blood flow through the occluded blood vessel. Revascularization techniques include using a variety of devices to pass through the occlusion to create or enlarge an opening through the occlusion. In some cases, lesions such as fibrotic and calcified lesions may create problems for revascularization techniques, and treatments to soften them and make them more malleable can be beneficial.

In some cases, for example, ultrasound may be used to treat vascular lesions, such as fibrotic and calcified lesions, at various states of disease progression, ranging from soft plaques to severely calcified lesions. Vascular lesions that may best lend themselves to being treated with ultrasound-based devices include irregular, severely calcified plaques that are located within and adjacent to vessel walls, and lesions that are more or less rigid and thus may be susceptible to being mechanically fatigued to failure. For example, ultrasound-based devices may be used to produce standing wave pressure patterns within the thickness of the lesion, bending moments at the ends of the lesion, and/or resonance along the length of the lesion. In some cases, the high frequency mechanical action of ultrasound may also be effective in treating earlier state vascular lesions, including fibrotic and soft plaques. In some cases, an ultrasound device may apply a treatment of unfocused, near-field ultrasound waves to treat vascular lesions.

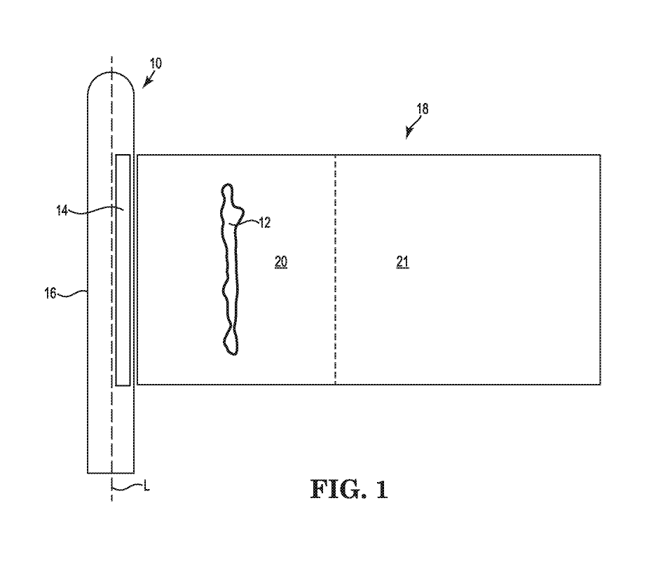

An intravascular device such as an ultrasound catheter may be placed within a blood vessel in order to treat a vascular lesion that is within or adjacent to a vessel wall of the blood vessel. FIG. 1 is a schematic view of an ultrasound catheter 10 placed proximate a calcified lesion 12. The ultrasound catheter 10 includes an ultrasound transducer 14 disposed relative to an elongate shaft 16. In some cases, the ultrasound transducer 14 may include a piezoelectric material, which transmits acoustic pressure in response to an applied voltage. The ultrasound transducer 14 may be driven at one or more frequencies in the range of about 20 kilohertz (kHz) to about 50 megahertz (MHz). The ultrasound transducer 14 may be a single ultrasound transducer, or the ultrasound transducer 14 may include a series of ultrasound transducers that may be operated to effectively function as a single ultrasound transducer, providing the desired acoustic pressure over the desired treatment area. The acoustic pressure applied may range from tens of kiloPascals (kPa) to in excess of ten megaPascals (MPa).

As can be seen in the example of FIG. 1, the ultrasound transducer 14 produces an ultrasound field 18 that includes a near field region 20 and a far field region 21. In the near field region 20, dynamic acoustic pressures may be cyclically applied to the calcified lesion 12. As used in this application, the near field region 20 refers to a region in close proximity radially to a surface of the ultrasound transducer 14, for example, the region extending outward from the transducer surface to a radial distance less than or equal to a length of the ultrasound transducer 14, wherein the acoustic pressure waves transmitted by the ultrasound transducer 14 are unfocused and can be controlled to be substantially uniform upon the calcified lesion 12.

In some cases, for example, the ultrasound transducer 14 may be configured to impart a uniform or substantially uniform acoustic pressure along the length of the calcified lesion 12. In cardiac vessel disease states, vascular lesions may span a length up to 50 millimeters (mm) in vessels that are 2 mm to 4 mm in diameter. In peripheral vessel disease states, vascular lesions may span a length of up to 200 mm in vessels up to 12 mm in diameter. Depending on the therapeutic applications, the ultrasound transducer 14 may be configured to impart a uniform or substantially uniform acoustic pressure over a length of about 10 mm to about 60 mm at a radial distance of about 1 mm to about 8 mm as measured from a central axis L extending through the elongate shaft 16. While not illustrated, one can appreciate that multiple ultrasound transducers 14 may be configured upon a catheter to extend the effective therapeutic length, such as up to a length of 200 mm.

To impart a uniform or substantially uniform acoustic pressure in the near field 20, the ultrasound transducer 14 may have a length that is multiple times larger than a diameter of the ultrasound catheter 10. In some cases, the ultrasound transducer 14 may have a length that is at least as long as a length of the calcified lesion 12. In some cases, the ultrasound transducer 14 may have a length that is about twice as long as a distance between the ultrasound transducer 14 and the calcified lesion 12, or about three times the distance, in some cases, to generate a uniform or substantially uniform acoustic pressure over a length of about 20 to about 80 mm.

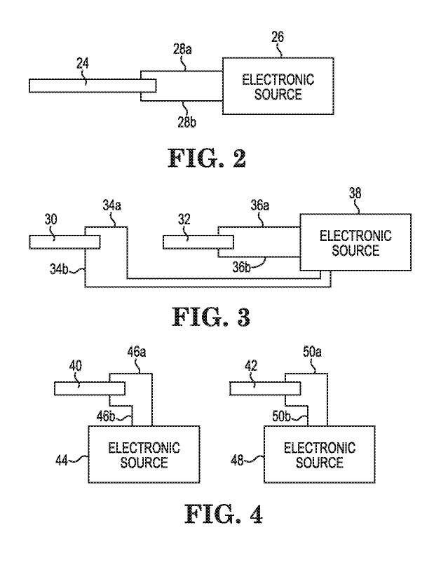

In some instances, the ultrasound transducer 14, may be a single ultrasound transducer or a series of ultrasound transducers or transducer elements driven in such a way as to effectively act as a single ultrasound transducer. FIGS. 2-4 provide illustrative but non-limiting examples of how the ultrasound transducer 14 may be controlled. In FIG. 2, a single ultrasound transducer 24 is electrically coupled to an electronic source 26 via wires 28a, 28b. FIG. 3 shows an ultrasound transducer 30 and an ultrasound transducer 32. The ultrasound transducer 30 is electrically coupled to an electronic source 38 via wires 34a, 34b and the ultrasound transducer 32 is electrically coupled to the electronic source 38 via wires 36a, 36b. In this case, the ultrasound transducer 30 and the ultrasound transducer 32 are driven with the same frequency and output from the electronic source 38. FIG. 4 shows an ultrasound transducer 40 and an ultrasound transducer 42. The ultrasound transducer 40 is electrically coupled to an electronic source 44 via wires 46a, 46b. The ultrasound transducer 42 is electrically coupled to an electronic source 48 via wires 50a, 50b. In this case, the ultrasound transducers 40, 42 are independently driven with the electronic sources 44, 48, respectively, and amplitude and phase control may be applied to increase the uniformity of the acoustic pressure imparted to the calcified lesion 12. While FIGS. 3 and 4 each show a pair of ultrasound transducers 30, 32 and 40, 42, it will be appreciated that this is merely illustrative, as any number of distinct ultrasound transducers may be utilized.

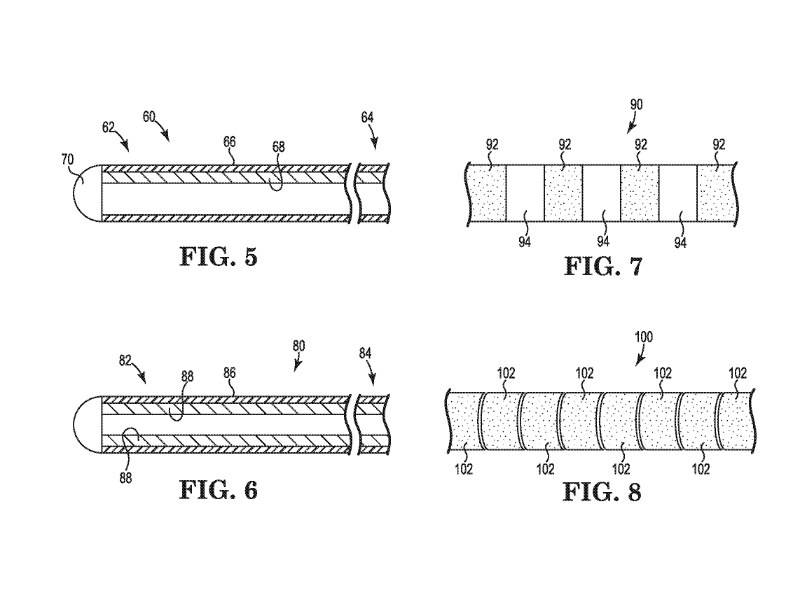

FIG. 5 is a schematic view of a portion of an ultrasound catheter 60 that includes a distal region 62 and a proximal region 64. The ultrasound catheter 60 includes an elongate shaft 66 and an ultrasound transducer 68 that is secured relative to the elongate shaft 66. An atraumatic tip 70 forms a distal end of the ultrasound catheter 60. The elongate shaft 66 and the atraumatic tip 70 may be formed of any suitable polymer, polymers, metal, metals, or a polymeric material over a metal. While the ultrasound transducer 68 is illustrated as being on an interior of the elongate shaft 66, in some cases, the ultrasound transducer 68 may instead be secured relative to an exterior of the elongate shaft 66. In some cases, the ultrasound transducer 68 may be a planar ultrasound transducer that is configured to transmit a highly directional ultrasound field with an axis of propagation orthogonal to a surface of the ultrasound transducer 68. In some cases, the ultrasound transducer 68 may occupy a greater portion of the catheter volume and may for example include greater piezoelectric material thickness, multiple matching layers, and backing layers to increase acoustic pressure output. While not illustrated, in some cases, the ultrasound catheter 60 may include a steering mechanism that allows a user to rotate the ultrasound catheter 60, or alternatively, rotate the ultrasound transducer 68 relative to the elongate shaft 66 in order to orient the ultrasound field in the desired direction.

FIG. 6 is a schematic view of a portion of an ultrasound catheter 80 that includes a distal region 82 and a proximal region 84. The ultrasound catheter 80 includes an elongate shaft 86 and an ultrasound transducer 88 that is secured relative to the elongate shaft 86. An atraumatic tip 70 forms a distal end of the ultrasound catheter 80. The elongate shaft 86 and the atraumatic tip 70 may be formed of any suitable polymer, polymers, metal, metals, or a polymeric material over a metal. While the ultrasound transducer 88 is illustrated as being on an interior of the elongate shaft 86, in some cases, the ultrasound transducer 88 may instead be secured relative to an exterior of the elongate shaft 86. In some cases, as illustrated, the ultrasound transducer 88 may include one or more cylindrical transducer elements that are configured to produce an ultrasound field emanating omnidirectionally with respect to a central axis of the ultrasound catheter 80.

FIG. 7 is a schematic view of a portion of an ultrasound catheter 90 including an ultrasound transducer including a number of individual ultrasound transducers or transducer elements 92 that are separated by flexible polymeric regions 94. The flexible polymeric regions 94 may be formed of any suitable polymeric material. It will be appreciated that separating the individual ultrasound transducers or transducer elements 92, which are rigid, with flexible polymeric regions 94 provides flexibility to the ultrasound catheter 90, which is useful in delivering the ultrasound catheter 90 through the vasculature to a target treatment site. In some cases, the number of individual ultrasound transducer elements 92 may be driven in such a way as to effectively function as a single ultrasound transducer, providing a uniform acoustic pressure upon a vascular lesion. In some cases, the flexible polymeric regions 94 may allow the ultrasound catheter 90 to assume a curvature of certain cardiac anatomy, such as but not limited to the right ventricular wall, and the individual ultrasound transducer elements 92 may be driven with phase and amplitude control in order to apply uniform acoustic pressure upon the vascular lesion.

FIG. 8 is a schematic view of a portion of an ultrasound catheter 100 having an ultrasound transducer including a number of individual articulating ultrasound transducers or transducer elements 102. While not illustrated, the individual articulating ultrasound transducers 102 may be disposed over a polymeric sheath. In some cases, the individual articulating ultrasound transducers 102 may be configured to pivot relative to each other, providing flexibility to the ultrasound catheter 100, which is useful in delivering the ultrasound catheter 100 through the vasculature to a target treatment site. In some cases, the articulating ultrasound transducer elements 102 may be driven in such a way as to effectively function as a single ultrasound transducer, providing a uniform acoustic pressure upon a vascular lesion. For example, in some cases the articulating ultrasound transducers 102 may allow the ultrasound catheter 100 to assume a curvature of certain cardiac anatomy, such as but not limited to the right ventricular wall, and the individual ultrasound transducer elements 102 may be driven with phase and amplitude control in order to apply uniform acoustic pressure upon the vascular lesion.

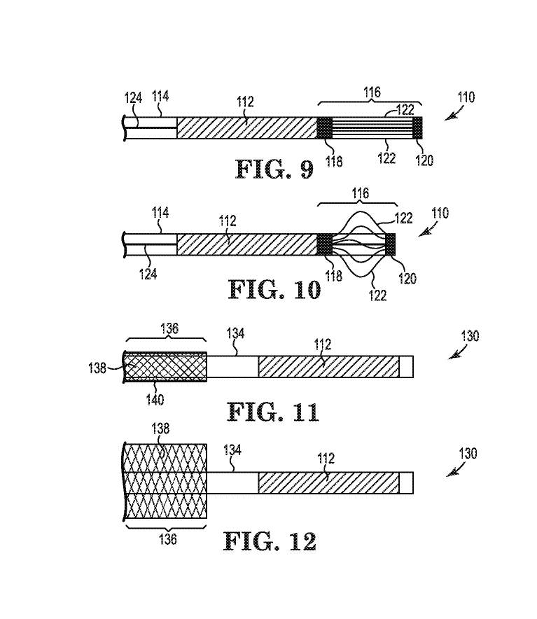

FIGS. 9 through 14 provide illustrative but non-limiting examples of fixation structures that may be used in combination with any of the ultrasound catheters 10, 60, 80, 90, 100 in order to temporarily anchor the ultrasound catheter 10, 60, 80, 90, 100 proximate a desired treatment site. The fixation structures may be considered as being movable between a collapsed configuration that permits the ultrasound catheter 10, 60, 80, 90, 100 to be advanced through a blood vessel to a desired treatment site and an expanded configuration that temporarily anchors the ultrasound catheter 10, 60, 80, 90, 100 at the desired treatment site. The fixation structures are subsequently movable back to the collapsed configuration for changing to a different treatment site, for example, or removing the ultrasound catheter 10, 60, 80, 90, 100.

FIGS. 9 and 10 show a distal portion of an ultrasound catheter 110 that includes an ultrasound transducer 112 disposed relative to an elongate shaft 114. The ultrasound transducer 112 may be considered as representing any of the ultrasound transducers 14, 24, 30, 32, 40, 42, 68, 88, 92, 102 described with respect to FIGS. 1-8. A fixation structure 116 includes a proximal anchor 118, a distal anchor 120 and a plurality of fixation elements 122 extending between the proximal anchor 118 and the distal anchor 120. A push/pull member 124 extends through the elongate shaft 114 and is operably coupled with the distal anchor 120. Comparing FIG. 9 with FIG. 10, it can be seen that in FIG. 10, the plurality of fixation elements 122 have expanded radially as a result of the push/pull member 124 being pulled proximally, thereby shortening the axial distance between the proximal anchor 118 and the distal anchor 120. After treatment, the ultrasound catheter 110 may be repositioned or withdrawn by extending the push/pull member 124 distally to collapse the plurality of fixation elements 122 to their original state.

FIGS. 11 and 12 show a distal portion of an ultrasound catheter 130 that includes an ultrasound transducer 112 disposed relative to an elongate shaft 134. The ultrasound transducer 112 may be considered as representing any of the ultrasound transducers 14, 24, 30, 32, 40, 42, 68, 88, 92, 102 described with respect to FIGS. 1-8. A fixation structure 136 includes a self-expanding mesh structure 138 and an outer sheath 140. As seen for example in FIG. 11, the outer sheath 140 constrains the expanding mesh structure 138 in a collapsed configuration for delivery and retrieval. Withdrawing the outer sheath 140 enables the expanding mesh structure 138 to self-expand into its expanded configuration, as seen in FIG. 12. After treatment, the outer sheath 140 or another structure may be advanced distally back over the expanding mesh structure 138 to return the expanded mesh structure 138 to its collapsed configuration for repositioning or removal.

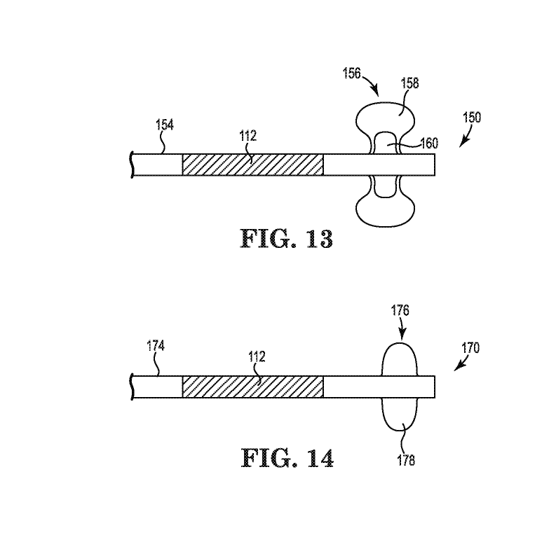

FIG. 13 shows a distal portion of an ultrasound catheter 150 that includes an ultrasound transducer 112 disposed relative to an elongate shaft 154. The ultrasound transducer 112 may be considered as representing any of the ultrasound transducers 14, 24, 30, 32, 40, 42, 68, 88, 92, 102 described with respect to FIGS. 1-8. A fixation structure 156 includes an inflatable balloon 158 and a channel 160 that is formed within the inflatable balloon 158. The inflatable balloon 158 may be deflated (not illustrated) for delivery, and may then be inflated to expand the inflatable balloon 158 into its expanded configuration (illustrated) in order to anchor the ultrasound catheter 150 in position. In some cases, the channel 160 extends through the inflatable balloon 158 and enables blood flow to pass through. As a result, the inflatable balloon 158, even when inflated, does not occlude blood flow through the blood vessel in which the ultrasound catheter 150 is disposed.

FIG. 14 shows a distal portion of an ultrasound catheter 170 that includes an ultrasound transducer 112 disposed relative to an elongate shaft 174. The ultrasound transducer 112 may be considered as representing any of the ultrasound transducers 14, 24, 30, 32, 40, 42, 68, 88, 92, 102 described with respect to FIGS. 1-8. A fixation structure 176 includes an inflatable balloon 178. The inflatable balloon 178 may be deflated (not illustrated) for delivery, and may then be inflated to expand the inflatable balloon 178 into its expanded configuration (illustrated) in order to anchor the ultrasound catheter 170 in position.

A variety of polymeric materials may be used in manufacturing the ultrasound catheters 10, 60, 80, 90, 100, 110, 130, 150, 170 described herein. Some examples of suitable polymers may include polytetrafluoroethylene (PTFE), ethylene tetrafluoroethylene (ETFE), fluorinated ethylene propylene (FEP), polyoxymethylene (POM, for example, DELRIN® available from DuPont), polyether block ester, polyurethane (for example, Polyurethane 85A), polypropylene (PP), polyvinylchloride (PVC), polyether-ester (for example, ARNITEL® available from DSM Engineering Plastics), ether or ester based copolymers (for example, butylene/poly(alkylene ether) phthalate and/or other polyester elastomers such as HYTREL® available from DuPont), polyamide (for example, DURETHAN® available from Bayer or CRISTAMID® available from Elf Atochem), elastomeric polyamides, block polyamide/ethers, polyether block amide (PEBA, for example available under the trade name PEBAX®), ethylene vinyl acetate copolymers (EVA), silicones, polyethylene (PE), Marlex high-density polyethylene, Marlex low-density polyethylene, linear low density polyethylene (for example REXELL®), polyester, polybutylene terephthalate (PBT), polyethylene terephthalate (PET), polytrimethylene terephthalate, polyethylene naphthalate (PEN), polyetheretherketone (PEEK), polyimide (PI), polyetherimide (PEI), polyphenylene sulfide (PPS), polyphenylene oxide (PPO), poly paraphenylene terephthalamide (for example, KEVLAR®), polysulfone, nylon, nylon-12 (such as GRILAMID® available from EMS American Grilon), perfluoro(propyl vinyl ether) (PFA), ethylene vinyl alcohol, polyolefin, polystyrene, epoxy, polyvinylidene chloride (PVdC), poly(styrene-b-isobutylene-b-styrene) (for example, SIBS and/or SIBS 50A), polycarbonates, ionomers, biocompatible polymers, other suitable materials, or mixtures, combinations, copolymers thereof, polymer/metal composites, and the like. In some embodiments the polymeric materials may include a liquid crystal polymer (LCP). For example, the mixture can contain up to about 6 percent LCP.

In some cases, the ultrasound catheters 10, 60, 80, 90, 100, 110, 130, 150, 170 may include a lubricious, a hydrophilic, a hydrophobic, a protective, or other type of coating. Hydrophobic coatings such as fluoropolymers provide a dry lubricity which improves device handling and device exchanges. Lubricious coatings improve steerability and improve lesion crossing capability. Suitable lubricious polymers are well known in the art and may include silicone and the like, hydrophilic polymers such as high-density polyethylene (HDPE), polytetrafluoroethylene (PTFE), polyarylene oxides, polyvinylpyrolidones, polyvinylalcohols, hydroxy alkyl cellulosics, algins, saccharides, caprolactones, and the like, and mixtures and combinations thereof. Hydrophilic polymers may be blended among themselves or with formulated amounts of water insoluble compounds (including some polymers) to yield coatings with suitable lubricity, bonding, and solubility. Some other examples of such coatings and materials and methods used to create such coatings can be found in U.S. Pat. Nos. 6,139,510 and 5,772,609, which are incorporated herein by reference.

The devices described herein may be formed, for example, by coating, extrusion, co-extrusion, interrupted layer co-extrusion (ILC), or fusing several segments end-to-end. The layer may have a uniform stiffness or a gradual reduction in stiffness from the proximal end to the distal end thereof. The gradual reduction in stiffness may be continuous as by ILC or may be stepped as by fusing together separate extruded tubular segments. The outer layer may be impregnated with a radiopaque filler material to facilitate radiographic visualization. Those skilled in the art will recognize that these materials can vary widely without deviating from the scope of the present invention.

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example embodiment being used in other embodiments. The scope of the disclosure is, of course, defined in the language in which the appended claims are expressed.

What is claimed is:

1. An ultrasound catheter adapted for placement within a blood vessel having a vessel wall, the ultrasound catheter for treating a calcified lesion within or adjacent the vessel wall, the ultrasound catheter comprising:
an elongate shaft extending from a distal region to a proximal region; and
an ultrasound transducer disposed within the distal region of the elongate shaft, the ultrasound transducer adapted to impart near-field acoustic pressure waves within the calcified lesion in order to induce fractures in the calcified lesion.

2. The ultrasound catheter of claim 1, wherein the ultrasound transducer is configured to transmit a substantially uniform acoustic pressure over a length of about 5 millimeters to about 60 millimeters at a radial distance of about 1 millimeters to about 8 millimeters as measured from a longitudinal central axis of the elongate shaft.

3. The ultrasound catheter of claim 1, wherein the ultrasound transducer is configured as a planar ultrasound transducer, and is adapted to output acoustic pressure waves propagating along a primary radial direction.

4. The ultrasound catheter of claim 1, wherein the ultrasound transducer is configured as a plurality of planar ultrasound transducers, and is adapted to output acoustic pressure waves propagating in a plurality of radial directions.

5. The ultrasound catheter of claim 1, wherein the ultrasound transducer is configured as a cylindrical ultrasound transducer, and is adapted to output acoustic pressure waves propagating radially outwardly, omnidirectionally from the cylindrical ultrasound transducer.

6. The ultrasound catheter of claim 1, wherein the ultrasound transducer comprises a plurality of individual ultrasound transducers.

7. The ultrasound catheter of claim 6, wherein the plurality of individual ultrasound transducers are axially spaced apart, with intervening polymeric segments disposed between adjacent ultrasound transducers to impart a degree of flexibility to the ultrasound transducer.

8. The ultrasound catheter of claim 6, wherein the plurality of individual ultrasound transducers are pivotably securable to one another to impart a degree of flexibility to the ultrasound transducer.

9. The ultrasound catheter of claim 6, wherein each of the individual ultrasound transducers are independently electrically driven.

10. The ultrasound catheter of claim 6, wherein all of the individual ultrasound transducers are electrically driven with a common source.

11. The ultrasound catheter of claim 1, further comprising a fixation structure coupled relative to the elongate shaft and moveable between a collapsed configuration that permits the ultrasound catheter to be advanced through a blood vessel and an expanded configuration that anchors the ultrasound catheter within the blood vessel.

12. The ultrasound catheter of claim 11, wherein the fixation structure is mechanically actuatable between the collapsed configuration and the expanded configuration.

13. The ultrasound catheter of claim 11, wherein the fixation structure is constrained in the collapsed configuration for delivery via an outer sheath disposed over the fixation structure and is self-expanding into the expanded configuration upon removal of the outer sheath.

14. An ultrasound device adapted for placement within a blood vessel having a vessel wall, the ultrasound device adapted for causing mechanical fractures in a calcified lesion within or adjacent the vessel wall, the ultrasound device comprising:
an elongate shaft extending from a distal region to a proximal region; and
an ultrasound transducer disposed within the distal region of the elongate shaft, the ultrasound transducer adapted to impart unfocused acoustic pressure waves within the calcified lesion in order to induce fractures in the calcified lesion;
the ultrasound transducer having an effective length that is at least twice a distance between the ultrasound transducer and the calcified lesion when the ultrasound device is disposed proximate the calcified lesion.

15. The ultrasound device of claim 14, wherein the ultrasound transducer has an effective length that is at least three times a distance between the ultrasound transducer and the calcified lesion when the ultrasound device is disposed proximate the calcified lesion.

16. The ultrasound device of claim 14, wherein the ultrasound transducer has an effective length that is longer than a length of the calcified lesion.

17. The ultrasound device of claim 14, further comprising a fixation element coupled to the elongate shaft and adapted to releasably secure the ultrasound device within a blood vessel.

18. An ultrasound catheter adapted for placement within a blood vessel having a vessel wall, the ultrasound catheter for treating a vascular lesion within or adjacent the vessel wall, the ultrasound catheter comprising:
an elongate shaft extending from a distal region to a proximal region;
a fixation structure coupled relative to the elongate shaft and moveable between a collapsed configuration that permits the ultrasound catheter to be advanced through a blood vessel and an expanded configuration that anchors the ultrasound catheter within the blood vessel; and
an ultrasound transducer disposed within the distal region of the elongate shaft, the ultrasound transducer adapted to impart near-field acoustic pressure waves upon the vascular lesion in order to mechanically modify the vascular lesion and increase distensibility of the blood vessel.

19. The ultrasound catheter of claim 18, wherein the fixation structure is mechanically actuatable between the collapsed configuration and the expanded configuration.

20. The ultrasound catheter of claim 18, wherein the fixation structure is self-expanding.
